(19) United States
(12) Patent Application Publication
(10) Pub. No.: US 2006/0264810 A1
(43) Pub. Date: Nov. 23, 2006

(54) Percutaneous respiratory assist catheter incorporating a spinning fiber bundle

(76) Inventors: Brack G. Hattler, Pittsburgh, PA (US); William J. Federspiel, Pittsburgh, PA (US); Brian J. Frankowski, Imperial, PA (US)

Correspondence Address: FULWIDER PATTON, 6060 CENTER DRIVE, 10TH FLOOR, LOS ANGELES, CA, 90045, US

(21) Appl. No.: 11/408,576

(22) Filed: Apr. 21, 2006

Related U.S. Application Data
(60) Provisional application No. 60/688,861, filed on Jun. 8, 2005.
(60) Provisional application No. 60/673,885, filed on Apr. 21, 2005.

(51) Int. Cl. A61M 37/00

(52) U.S. Cl. 604/26

(57) ABSTRACT

A compact,intravenous, percutaneous respiratory assist catheter having a rotating fiber bundle functions as an intravenous oxygenerator configured to be implanted within the patient's vasculature. The respiratory assist catheter provides oxygen introduction and carbon dioxide removal from the blood of the patient. the catheter includes hollow, gas-permeable fibers extending between a distal manifoldand a proximal manifold that permit diffusion of gases between the blood vessel and the interior of the fibers. An implantable version of the catheter is configured with a fiber bundle having increased porosity and with a mechanism to prevent the fiber bundle from damaging the vena cava. The fiber bundle may be protected by a wire loom or coil cage made from materials such as nitinol and stainless steel. The rotation of the fiber bundle may be varied in speed and in direction.

### CROSS-REFERENCES TO RELATED APPLICATIONS

This application claims the benefit of U.S. Provisional Application Ser. No. 60/688,861, filed Jun. 8, 2005 and U.S. Provisional Application Ser. No. 60/673,885, filed Apr. 21, 2005, the contents of which are hereby incorporated herein by reference.

### FEDERALLY SPONSORED RESEARCH

The U.S. Government has a paid-up license in this invention and the right in limited circumstances to require the patent owner to license others on reasonable terms as provided for by the terms of Grant No. DAMD 17-98-1-8638 awarded by the Department of the Army, and Grant No. R01 HL 70051 by the National Institutes of Health.

### BACKGROUND OF THE INVENTION

The present invention relates generally to the field of intravenous oxygenators used to increase the oxygen level and decrease the carbon dioxide content in a patient's blood. More specifically, the present invention is directed to a percutaneous respiratory assist catheter having a rotating or spinning fiber bundle that enhances the exchange of gases between the oxygenator and the surrounding blood.

Many types of blood oxygenators are known in the art. For example, during open heart surgery, the patient is interconnected with an external oxygenator, commonly known as a heart-lung machine, which introduces oxygen into the blood system. Most types of oxygenators use a gas-permeable membrane. Blood flows along one side of the membrane and oxygen is supplied to the other side of the membrane. Given a sufficient partial pressure gradient between the oxygen supply and the blood, oxygen will diffuse through the membrane and into the blood. In addition, carbon dioxide in the blood will tend to diffuse from the blood into the interior of the membrane.

In other situations, a smaller implantable oxygenator may be sufficient to adequately supplement the patient's cardiopulmonary function by marginally increasing the oxygen content of the patient's blood. For example, patients suffering from emphysema, pneumonia, congestive heart failure, or other chronic lung disease often have arterial blood oxygen partial pressures of approximately forty torr. A relatively small increase of ten percent to twenty percent is generally sufficient to adequately maintain the patient. An implantable oxygenator, such as a respiratory support catheter, is a particularly desirable alternative in that it avoids the need to intubate the patient in such cases. In addition, temporary use of an implantable oxygenator is sufficient in many cases to tide the patient over an acute respiratory insult. Placing such patients on a conventional respirator is often the beginning of a progressive downhill spiral by damaging the patient's pulmonary tree and thereby causing greater dependence on the respirator.

Implantable oxygenators, respiratory assist catheters and respiratory support catheters typically include a plurality of hollow gas-permeable membrane fibers that form a loop or are woven into a mat so that oxygen or other so-called “sweep gas” can be fed into one end of each fiber. Carbon dioxide enriched sweep gas is removed from the other end of the fibers as a result of the cross-diffusion that takes place. The effective rate of diffusion in implantable oxygenators can be limited in some instances by the problem of “streaming” or “channeling”, wherein the blood stream establishes relatively stable patterns of flow around and through the oxygenator. Only portions of the fibers are exposed to a relatively high velocity, turbulent flow of blood. These conditions tend to increase cross-diffusion of oxygen and carbon dioxide. However, other portions of the fibers are exposed to a low velocity, laminar flow of blood that reduces diffusion of gases. Those portions of the fibers immediately adjacent to the regions of high blood flow may continue to experience high rates of diffusion, but the remaining portions of the fibers tend to have relatively low diffusion rates. Thus, the overall diffusion rate of the oxygenator can be substantially diminished by streaming.

A prior art respiratory support catheter has been disclosed previously that is configured with a plurality of hollow gas-permeable but liquid-impermeable fibers that are formed into loops and are configured to be inserted into a blood vessel. High oxygen content sweep gas is fed into one end of the fibers and carbon dioxide laden sweep gas is withdrawn from the opposite end of the fibers. Oxygen and carbon dioxide diffuse through the fiber walls when the fiber loops of the catheter are positioned within the blood vessel. In one embodiment of the prior art respiratory support catheter, a system for agitating the blood is positioned within the loops formed by the fibers so that the linear flow of blood is disrupted and the blood is directed radially by the agitator to randomly move the fibers and thereby prevent streaming. For example, the disclosure included an agitator having a rotating curved blade designed to disrupt the linear blood flow and redirect the flow into swirling radially-oriented patterns.

Accordingly, there is a need for, and what was heretofore unavailable, a percutaneous respiratory assist catheter having enhanced gas exchange characteristics resulting from a rotating or spinning fiber bundle, including increased porosity of the fiber bundle and improved protection of the vasculature from the spinning components of the device where applicable.

### SUMMARY OF THE INVENTION

The present invention is directed to a compact, intravenous, percutaneous respiratory assist catheter that increases gas exchange efficiency and hence reduces size by incorporating a rotating hollow fiber bundle. The spinning fiber bundle provides an increase in velocity of the fluid relative to the fibers and a larger relative velocity than would otherwise exist in the vena cava in the absence of this fiber rotation. In this configuration the device can achieve gas exchange levels two to three times higher than respiratory catheters based on balloon pulsation.

The present invention provides an intravenous oxygenator configured as a respiratory assist catheter having a plurality of hollow gas-permeable fibers extending between a proximal manifold and a distal manifold. The respiratory support catheter may include a hollow rotatable central shaft extending from the proximal manifold and into the distal manifold. Oxygen may be supplied to the distal manifold via the central shaft, and a vacuum pump may be connected to the proximal manifold so as to induce flow of oxygen through the fibers. Alternatively, oxygen can be supplied through the proximal manifold and suction can be applied via the central shaft and distal manifold. The oxygenator is inserted into a blood vessel so that when oxygen is drawn through the fibers, it will diffuse through the walls of the fibers and into the adjacent blood stream, while excess carbon dioxide in the blood will pass in a reverse or cross-diffusion pattern through the walls of the fibers into the interior thereof for removal from the fibers.

The present invention includes a percutaneous respiratory assist catheter having a fiber bundle fabricated utilizing hollow fiber membranes configured around a stainless steel shaft. The proximal end of the shaft extends out of the fiber bundle and is configured to connect to a motor configured to rotate or spin the fiber bundle. The proximal and distal ends of the fiber bundle are potted to form manifolds creating a single gas pathway. The potted manifolds are sealed in bearing housings to separate the gas from the liquid. In use, oxygen and carbon dioxide are exchanged with the surrounding fluid (i.e., blood in vivo) during rotation of the fiber bundle.

The respiratory assist catheter of the present invention may be configured to increase the porosity in the rotating fiber bundle. The increased porosity provides more fluid to flow through the fiber bundle, thus increasing the overall mass transfer efficiency of the device. The extra porosity in the fiber bundle is created by several possible ways including, but not limited to, using spacers to create void space between the fiber layers, removing every other fiber in the mat and using smaller diameter fibers. Additionally, support threads could be removed from the fiber fabric, and the respiratory assist catheter could be configured such that the manifolds are relatively closer so as to “puff out” the fiber bundle.

The present invention includes a method of inserting the respiratory assist catheter into the vasculature of a patient, operating the device so as to facilitate introduction of oxygen to and removal of carbon dioxide from the patient's blood stream. The distal portion of the respiratory assist catheter may be implanted in the venous system of the patient through a single small incision. For example, the device can be implanted through the right femoral vein or internal jugular vein and guided into the superior vena cava and right atrium of the patient. For maximum effectiveness, the fiber bundle is placed in or proximate to the vena cava. Insertion of the oxygenator can be aided by using a conventional or specially configured introducer similar to the type presently employed to insert a stent, stent graft, cardiac pacemaker, etc. After oxygen and carbon dioxide levels are achieved and/or the patient is able to maintain sufficient gas exchange levels without assistance, the respiratory assist catheter is withdrawn from the vasculature.

Another aspect of the present invention includes a percutaneous respiratory assist catheter having the following features:
Catheter made from bundle of hollow fiber membranes
Inserted in femoral vein and placed within vena cava
Supply O2, remove CO₂ before blood reaches lung: partial respiratory support
Size reduction from known respiratory support catheters
The catheter has an insertion size of about a 20-25 French
Increased gas exchange efficiency from known respiratory support catheters
Catheters of 20-25 French having vCO₂ of about 50-70 ml/min
Cross section diameter about 11 mm
Fiber bundle length about 20 cm
Unsteady rotation of the fiber bundle, e.g., oscillatory rotation
Vanes or other devices incorporated into the manifolds for mixing blood

Other features and advantages of the invention will become apparent from the following detailed description, taken in conjunction with the accompanying drawings, which illustrate, by way of example, the features of the invention.

### BRIEF DESCRIPTION OF THE DRAWINGS

FIG. 1 depicts a plan view in partial cross-section of one embodiment of the respiratory assist catheter of the present invention.

FIG. 2 is a cross-sectional view of the respiratory assist catheter of FIG. 1 taken along lines 2-2.

FIG. 3 is a cross-sectional view of the respiratory assist catheter of FIG. 1 taken along lines 3-3.

FIG. 4 is a cross-sectional view of the respiratory assist catheter of FIG. 1 taken along lines 4-4.

FIG. 5 depicts a side plan view of an alternative embodiment of the respiratory assist catheter of the present invention.

FIG. 6 is a cross-sectional view of the respiratory assist catheter of FIG. 5 taken along lines 6-6.

FIG. 7 are graphical representations of gas exchange data in water and in blood using the respiratory assist catheter of the present invention.

FIG. 8 depicts a perspective view of an alternative embodiment of the respiratory assist catheter of the present invention having a wire cage.

FIG. 9 depicts a perspective view of an alternative embodiment of the respiratory assist catheter of the present invention having a coil cage.

FIG. 10 depicts a schematic representation of a fiber mat having spacers for use in the respiratory assist catheter of the present invention.

### DETAILED DESCRIPTION OF THE PREFERRED EMBODIMENTS

As shown in the drawings for purposes of illustration, the present invention is directed to a percutaneous respiratory assist catheter having a rotating or spinning fiber bundle fabricated utilizing hollow fiber membranes. U.S. Pat. Nos. 4,911,689 (Hattler); 4,986,809 (Hattler); 5,122,113 (Hattler); 5,207,640 (Hattler); 5,219,326 (Hattler); 5,271,743 (Hattler); 5,376,069 (Hattler); 5,501,663 (Hattler et al.) and 5,865,789 (Hattler) are incorporated herein in their entirety by reference.

The respiratory assist catheter of the present invention is configured with hollow, gas-permeable fibers extending between a distal. manifold and a proximal manifold that permit diffusion of gases between the patient's blood and interior of the fibers. A rotatable support member extends through the proximal manifold and into the distal manifold. The catheter includes bearings and seals within or adjacent to the proximal and distal manifolds. The support member may include a lumen in communication with the distal manifold so that oxygen-containing gases flow through the support member, distal manifold, fibers, and proximal manifold. The respiratory assist catheter may include a coupling at the proximal end of the support member for connecting the catheter to a supply of high oxygen (O₂) content sweep gas. The proximal coupling may also include a port for connecting the proximal manifold to a vacuum line to remove the carbon dioxide (CO₂) enriched sweep gas. The proximal coupling may also serve as a handle for the physician to manipulate the catheter during introduction of the distal portion of the respiratory assist catheter into the patient's vasculature. A metal, wire or similar shaft may extend from the distal portion of the catheter through the proximal manifold, seals and bearings and extend past the proximal coupling (handle) and connect to a motor to provide rotation of the fiber bundle.

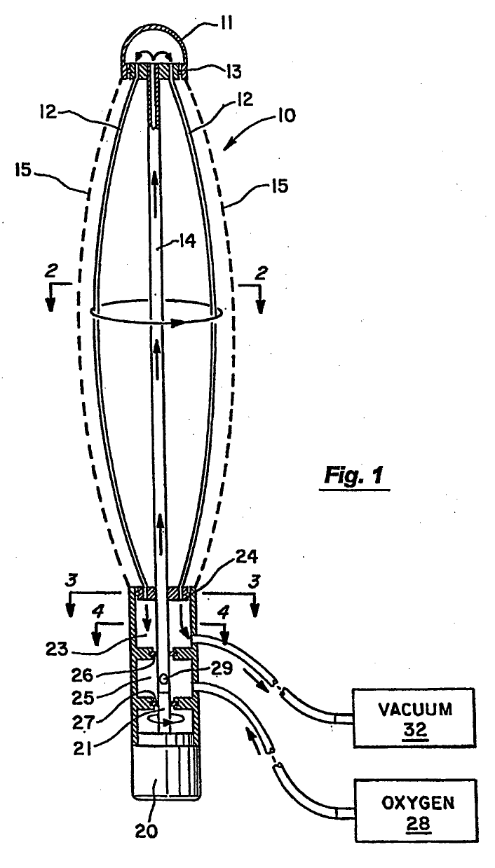

Referring now to FIG. 1, the present invention includes an intravenous oxygenator 10 having a plurality of hollow gas-permeable fibers 12. One end of each fiber 12 is potted into a distal manifold 11 and the other end of the fibers is potted into a proximal manifold 23, so that gas can flow between the manifolds 11 and 23 through the fibers 12. However, the ends of each fiber 12 are sealed in fluid communication with the manifolds 11 and 23 so that no gas can escape directly into the surrounding blood stream.

The gas-permeable walls of the fibers 12 provide a large total surface area for diffusion of oxygen into the blood stream, and for diffusion of carbon dioxide out of the blood stream. Any of a variety of flexible, hollow, gas-permeable fibers currently available on the market, such as Mitsubishi KPF-190 M polypropylene fibers, are suitable for this purpose. The polypropylene fibers may be coated with an ultra-thin coating of a gas-permeable polymer (e.g., silicone rubber having a thickness of less than 1 micron) and bonded with a non-thrombogenic component (e.g., heparin).

The distal manifold 11 can be molded from plastic or rubber around the ends of the fibers 12 to prevent the escape of gases at the junction between the fiber ends and the distal manifold 11. For example, as shown in FIG. 1, the distal manifold 11 can be formed as a tapered tip that is contoured to ease insertion of the oxygenator 10 through an incision. The proximal manifold 23 is shown in the cross-sectional view provided in FIG. 4. A vacuum pump 32 can be connected to the vacuum port 30 of the proximal manifold 23, as illustrated in FIG. 1, to enhance the flow of gases through the fibers 12.

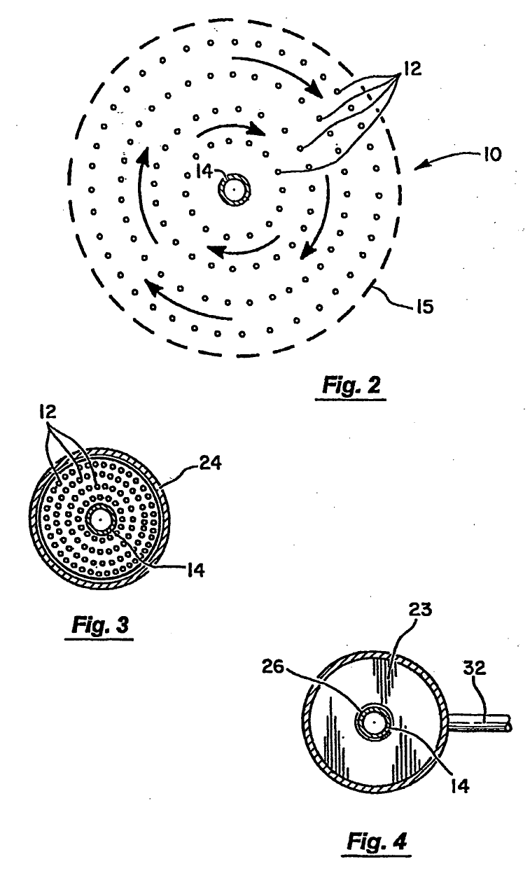

A hollow, rotatable, central shaft 14 extends through the proximal manifold 23 and then passes into the interior of the distal manifold 11. The central shaft 14 has at least one hollow lumen extending along its entire length that allows oxygen to be distributed through the central shaft 14 to the distal manifold 11 as illustrated in FIG. 1. A cross-sectional view of the upper portion of the oxygenator 10 is provided in FIG. 4. In the preferred embodiment of the present invention, the fibers 12 rotate with the central shaft 14. FIG. 2 is a cross-sectional view of the rotating central shaft 14 and fibers 12. As illustrated in FIG. 3, a sealing ring 24 on the exposed end of the proximal manifold 23 allows the proximal ends of the fibers 12 to freely rotate with the central shaft 14, while preventing the escape of gases from within the proximal manifold 23. Another sealing ring 13 on the underside of the distal manifold 11 allows the distal ends of the fibers 12 to freely rotate with the central shaft 14 while maintaining a gas-tight seal. These sealing rings 13 and 24 allow the fibers 12 to rotate with the central shaft 13, but permit the distal tip 11 to remain relatively stationary and thereby reduce the risk of trauma to the blood vessel.

In one embodiment of the present invention, the fibers 12 are formed into a plurality of flat mats. For example, the fiber mats can be wound concentrically, helically, or in some other radially-symmetric pattern about the central shaft 14.

A porous cage or enclosure 15 extends between the distal manifold 11 and proximal manifold 23, and surrounds the fibers 12 as illustrated in FIGS. 1 and 2. This cage 15 protects the lining of the blood vessel from the spinning fibers 12.

The proximal end of the central shaft 14 extends through the proximal manifold 23, as shown in FIG. 4, and is connected by a motor 20, as shown in FIG. 1. The motor 20 rotates the central shaft 14, which in turn spins the distal tip 11 and fibers 12 to create turbulent blood flow. In the preferred embodiment, the central shaft 14 spins at high velocity (e.g., up to 6,000 to 10,000 revolutions per minute). The resulting rotation of the fibers 12 improves the distribution of blood within the fibers 12 and enhances the exchange of oxygen and carbon dioxide.

To summarize, oxygen-containing gases flow from an external supply through the central shaft 14, into the distal manifold 11, through the fibers 12, and are then exhausted through the proximal manifold 23. Thus, the central shaft 14 serves both to: (1) act as the axis for supporting and spinning the fibers 12; and (2) provide a lumen for delivering oxygen to the distal manifold 11. The central shaft 14 also acts as a structural support for the distal manifold 11 and fibers 12, and provides a degree of rigidity to aid initial insertion of the oxygenator 10 into the blood vessel.

The lower portion of FIG. 1 depicts the assembly used to simultaneously drive the central shaft 14 and supply oxygen. The proximal end of the central shaft 14 is enclosed in an oxygen supply manifold 25. Oxygen is supplied from an oxygen source 28 through the oxygen supply manifold 25 via a number of small openings 29 into the lumen of the central shaft 14. The lower end of the central shaft 14 passes through two seals 26 and 27 in the top and bottom walls, respectively, of the oxygen supply manifold 25 that allow the central shaft 14 to freely rotate when driven by the motor 20. Alternatively, a short cable or a separate shaft could be interposed between the motor 20 and the central shaft 14.

A number of alternative embodiments and additional features are possible. For example, the previous discussion assumes that the motor 20 runs continuously to spin the fibers either in a clockwise or counter-clockwise direction. However, the motor could be rapidly cycled in alternating directions to oscillate the central shaft 14 and fibers 12. If the range of rotary motion is limited to a fraction of a complete revolution, it might be possible to omit the sealing ring 24 on the proximal manifold 23.

In another embodiment, the central shaft can be replaced with a hollow, flexible cable 141. The flexible cable 141 has a substantially air-tight central lumen that supplies oxygen to the distal manifold 11 in the same manner as previously described. Thus, either a rigid central shaft 14 or a flexible cable 141 could be employed as a support member to provide structural support for the distal manifold 11, to rotate the fibers 12, and to deliver oxygen to the distal manifold 11. This flexibility simplifies insertion of the oxygenator 10 into a blood vessel and helps to minimize patient trauma.

In use, the distal portion of the oxygenator 10 is implanted in the venous system of the patient through a single small incision. For example, the device 10 can be implanted through the right internal jugular vein into the superior vena cava and right atrium of a patient. For maximum effectiveness, the distal manifold 11 and fibers 12 are fully inserted through the incision up to the level of the proximal manifold 23. Insertion of the oxygenator 10 can be aided by using a conventional introducer similar to the type presently employed to insert a cardiac pacemaker.

After the device has been implanted, a supply of oxygen-containing gas is connected to the oxygen supply manifold 25 via source 28. The oxygen flows through lumen of the central shaft 14 into the distal manifold 11 and through the fibers 12. Oxygen flows along the interior passageways of the fibers 12 and diffuses outward through the gas-permeable walls of the fibers 12 into the surrounding blood stream. Carbon dioxide also diffuses inward from the blood stream through these gas-permeable walls into the interior of the fibers 12. Carbon dioxide and any remaining oxygen in the fibers are vented to the atmosphere at the proximal ends of the fibers through the proximal manifold 23. Negative pressurization can be applied by means of a suction pump 32 connected to the proximal manifold 23 to enhance gas flow through the fibers 12.

In addition, after the oxygenator 10 has been inserted into the blood vessel up to the location of the proximal manifold 23, the motor 20 is energized to start the central shaft 14 rotating so that rotation of the fibers 12 will disrupt the linear flow of the blood and move the blood radially in swirling convective flow patterns to optimally expose the surface area of the fibers to the blood and maximize the cross diffusion of gases in and out of the fibers 12.

In a preferred use of the device, oxygen is introduced to the fibers 12 at a flow rate of approximately 1 to 3 liters per minute and at a nominal pressure of approximately 6 to 125 mmHg. A suction pressure of approximately −150 to −250 mmHG is applied at the proximal manifold 23. An alternate configuration would allow the oxygen to be delivered to the proximal manifold and the vacuum to be delivered at the distal manifold.

It should be noted that the present invention can also be used to administer anesthetic and other gases, such as nitric oxide (NO) and carbon monoxide (CO), or other medications directly into the patient's blood system. For this purpose, a mixture of oxygen and such gases or medications gases flow through the fibers 12 and diffuse into the patient's blood stream.

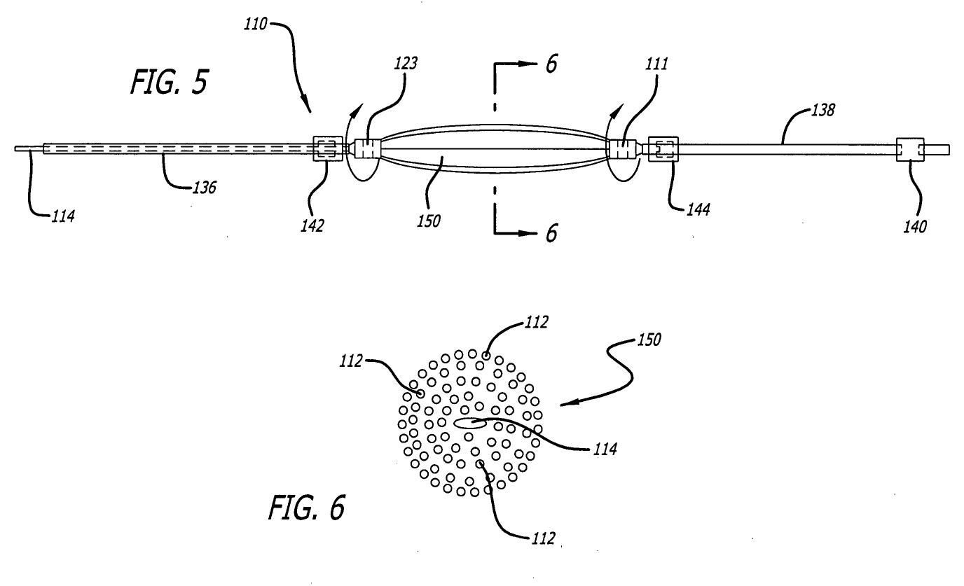

As shown in FIGS. 5 and 6, a bench prototype of the respiratory assist catheter 110 was constructed using a plurality of gas-permeable fibers 112 woven into a mat 150 around a actuator shaft 114. The proximal end of the actuator shaft extends out of the fiber bundle and is configured to connect to a motor 120. The actuator shaft may be formed from stainless steel or other suitable material having a diameter of about 0.037 centimeters (cm) and having a length of about thirty centimeters. The fiber bundle is capable of spinning from up to at least 10,000 rpm. To enhance performance of the gas exchange achieved by the respiratory assist catheter, various modes of spinning of the fiber bundle may be employed, e.g., steady rotation, unsteady rotation, purely oscillatory rotation and other forms of time-dependent rotation. As will be appreciated by those of ordinary skill in the art, known and to-be-developed gas-permeable fibers may be used with the present invention, for example, hollow micro-porous polypropylene fibers and gas-permeable fibers currently used in blood oxygenators. The gas-permeable fibers may include a coating of a gas-permeable polymer and may be bonded with a non-thrombogenic component.

The ends of the fiber bundle 150 are potted to form a proximal manifold 123 and a distal manifold 111 creating a single gas pathway. The potted manifolds are sealed in bearing housings 142, 144 to separate the gas from the liquid. In use, oxygen and carbon dioxide are exchanged with the surrounding fluid (i.e., blood in vivo) during rotation of the fiber bundle. A sweep gas delivery catheter 136 surrounds the actuator shaft and is configured in fluid communication with the proximal manifold 123 so as to deliver oxygen laden gas to the fiber bundle. A sweep gas exhaust catheter 138 is configured in fluid communication with the distal manifold 111 to provide an exit flow of the carbon dioxide enriched gas, and may include a coupling 160 for connecting to a vacuum source (not shown). The proximal and distal manifolds may be configured with mechanisms, such as vanes, to aid in the mixing of fresh blood into the spinning fiber bundle.

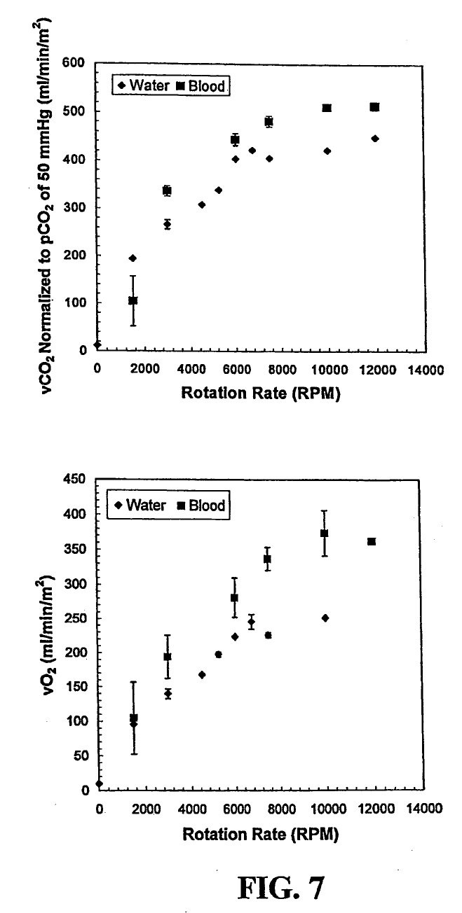

The prototype respiratory assist catheter 110 was configured with a fiber bundle 150 having an outside diameter of about 25 French and about 0.1 square meters (m²) of membrane surface area. The catheter was designed so that it could be steadily rotated up to 10,000 revolutions per minute (rpm). The performance of the rotating bundle catheter was characterized in a mock vena cava loop using water as the test fluid. The carbon dioxide (CO₂) and oxygen (O₂) gas exchange rates increased with increasing rate of bundle rotation exhibiting a plateau above 6000-7000 rpm. Gas exchange rates per unit membrane area achieved were 529±4.1 and 263±2.3 ml/min/m² for CO₂ and O2 , respectively, which were over two-fold greater than achieved in the same test using control respiratory catheters based on a pulsating balloon design of a respiratory support catheter. Bench tests using bovine blood indicated that the rotation of the fiber bundle per se does not appear to cause significant red cell hemolysis. As shown in FIG. 7, experiments using the prototype respiratory assist catheter resulted in some degrees of plateau in gas exchange with increasing rotation rate, which may be the result of the conditions under which the experiments were conducted.

An implantable embodiment of the respiratory assist catheter may include a cage or housing to protect the surrounding vessels from contacting rotating fibers, for example, to protect the vena cava from damage caused by the rotating fiber bundle. In vivo, the hollow fibers of the respiratory assist catheter are configured into a bundle, which is rotated about a central axis. Accordingly, there is potential for the fibers to contact the vasculature and damage the endothelial cells and other tissue. The present invention contemplates several embodiments of the protective cage for the respiratory assist catheter that will protect the device and the vena cava and include, but are not limited to, a wire loom cage, a coil cage and a laser cut cage. The present invention also includes devices and methods for the expansion and contraction of the cage mechanisms to facilitate insertion and removal of the device.

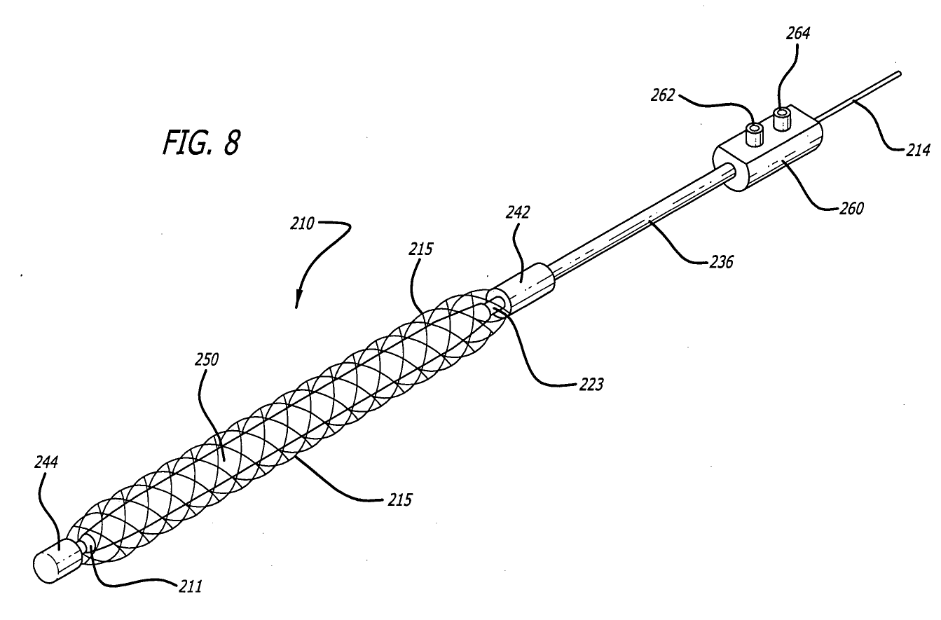

As shown in FIG. 8, the respiratory assist catheter 210 includes a wire loom cage 215 that may be manufactured from multiple strands of nitinol wire or other suitable material. For example, the wire loom cage may be manufactured in its expanded form by weaving nitinol wires over a mandrel. The nitinol cage may be heat treated to achieve the memory shape of the mandrel. The cage is attached to the proximal and distal ends of the non-rotating components of the respiratory assist catheter. For example, the proximal end of the cage may be fixedly attached to a proximal coupling 242 that is in rotatable engagement with the proximal manifold 223 of the catheter. Similarly, the distal end of the cage may be fixedly attached to a distal coupling 244 that is in rotatable engagement with the distal manifold 221 of the catheter.

A multi-lumen catheter shaft 236 is configured in fluid communication with the proximal manifold 223 and/or distal manifold 211 of the respiratory support catheter 210. The proximal end of the catheter shaft may include a handle or other coupling 260 having a sweep gas inlet (O₂) port 262 and a sweep gas exhaust (CO₂ - vacuum) port 264. The catheter shaft and handle may be made from various material well known to those of ordinary skill in the art, such as PEBAX. The catheter shaft may house an actuator shaft 214 formed from stainless steel or other suitable material. The proximal end of the actuator shaft may extend out of the handle of the catheter shaft and may be coupled with a motor or other mechanism (not shown) configured to spin or rotate the actuator shaft, which is fixedly attached to the fiber bundle 250.

The wire loom cage 215 may be compressed with a sheath (not shown) made from polytetrafluoroethylene (PTFE) or other suitable polymer, plastic, elastomer or biocompatible material. The cage is configured to expand by removing the sheath, and the cage is configured to constrict by replacing the sheath around the cage. The wire loom cage is configured to stand free around the fiber bundle 250 to protect surrounding tissue from contacting the rotating fibers without the rotating fiber bundle contacting the wires of the cage.

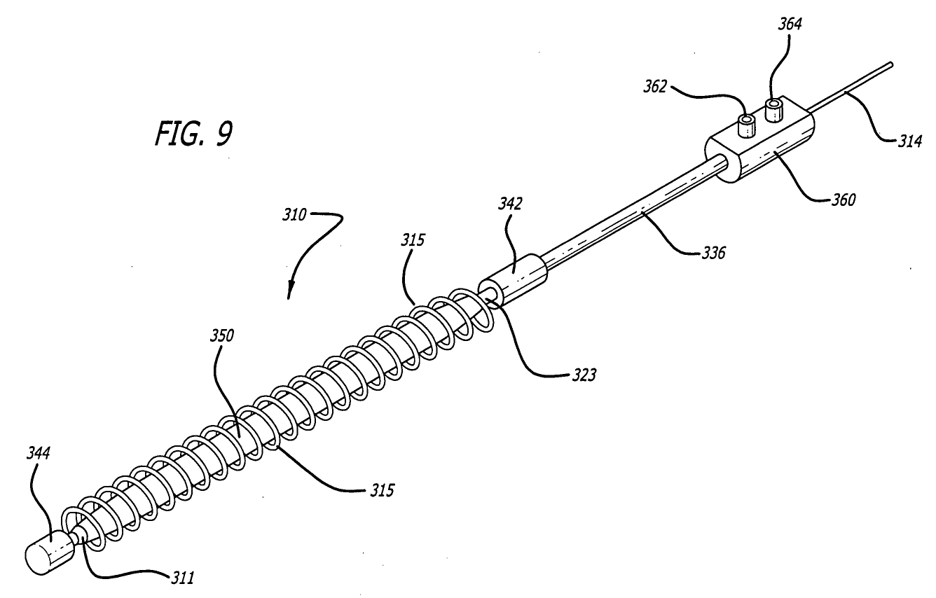

As shown in FIG. 9, a coil cage 315 is configured as a spring that can be expanded or contracted over the fiber bundle 350 of the respiratory assist catheter 310 by rotating the proximal end of the cage either clock-wise to expand or counter clock-wise to contract the coil about the fiber bundle. The coil is attached to the proximal 323 and distal 311 ends of the non-rotating components of the respiratory assist catheter 336 to protect the vena cava from contacting the rotating fibers. The coil cage is configured to expand or contract around the fiber bundle without interfering with the rotation of the fiber bundle. In addition, a laser cut cage (not shown) may be manufactured from a solid thin wall nitinol tube or other suitable material, such as stainless steel. Such a laser cut cage is attached to the proximal and distal ends of the non-rotating components of the respiratory assist catheter. The cage may be compressed with a PTFE or other suitable sheath. The laser cut nitinol cage is configured to expand by removing the sheath, and configured to contract by replacing the sheath around the cage. The laser cut cage is configured to stand free around the fiber bundle to protect surrounding tissue from contacting the rotating fibers, and to allow the fiber bundle to rotate without contacting the cage. The laser cut cage may be made in its expanded form using the well known technology applied to manufacturing abdominal medical devices, such as coronary, peripheral and abdominal aortic aneurism (AAA) stents. Accordingly, U.S. Pat. No. 5,780,807 is incorporated herein in its entirety by reference.

In a further embodiment of the present invention, the respiratory assist catheter is configured to increase the porosity in the rotating fiber bundle. The increased porosity provides more fluid to flow through the fiber bundle, thus increasing the overall mass transfer efficiency of the device. The extra porosity in the fiber bundle is created by several possible ways including, but not limited to, using spacers to create void space between the fiber layers, removing every other fiber in the mat and using smaller diameter fibers. Additionally, support threads could be removed from the fiber fabric, and the respiratory assist catheter could be configured such that the manifolds are relatively closer so as to “puff out” the fiber bundle.

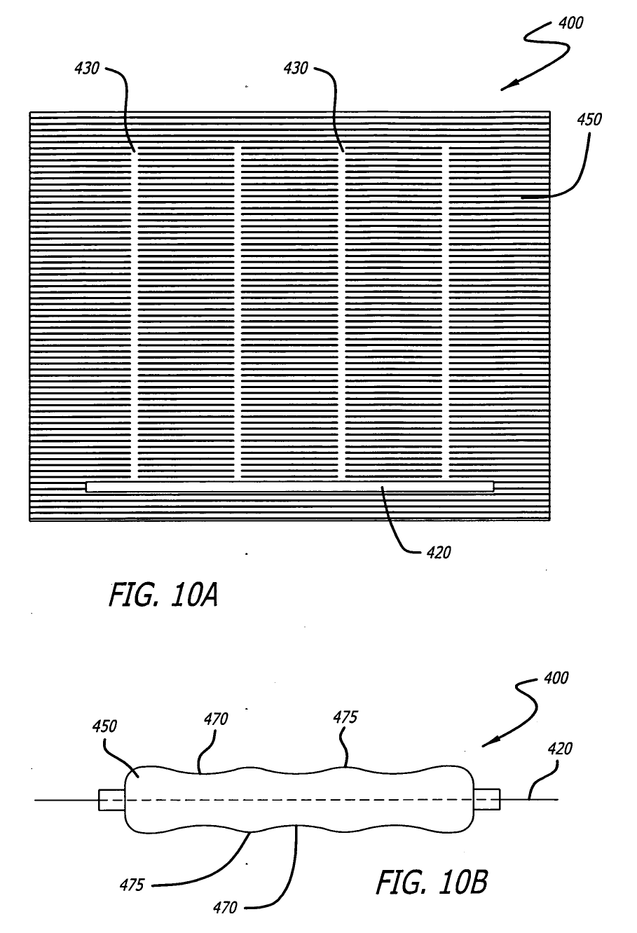

As shown in FIGS. 10A and 10B, A fiber bundle 400 may be configured with spacers 430 can be created by placing thin strips of felt that are soaked in polyurethane or other suitable material across a fiber mat 450. In accordance with the present invention, as the individual fibers 450 of the fiber mat are rolled up, the felt is rolled with it, which then hardens as the adhesive dries. The dried felt then creates the extra space between the fibers, creating hills 475 and valleys 470 in the rolled mat (FIG. 10B). However, the fiber surface area where the felt is touching is not included in the operable surface area of the respiratory assist catheter. The fiber bundle may include a stabilizing rod 420 within the inside of the rolled fiber mat.

Alternatively, by removing every other fiber in the fiber mat, the fiber mat is left with many open spaces having only wefts and no fibers. The same overall surface area and number of fibers may be the same, but the fibers are much more spaced out, thus creating a “puffy bundle.” Further, smaller diameter fibers can also be used to create higher porosity devices. The porosity is higher because the fiber density in a mat configured with smaller outer diameter fibers is less than fiber mats configured with fibers having larger outer diameters. There is much more open space where only wefts exist, similar to configurations of the fiber bundle where every other fiber is removed.

While a particular form of the invention has been illustrated and described, it will also be apparent to those skilled in the art that various modifications can be made without departing from the spirit and scope of the invention. Additional features of the respiratory assist catheter of the present invention may be found in FIGS. 11 and 12. Accordingly, it is not intended that the invention be limited by the specific embodiments disclosed herein.

We Claim:

1. A percutaneous respiratory assist catheter, comprising:
a catheter having a plurality of lumens;
a bundle of hollow fiber membranes connected to the catheter and in fluid communication with at least one lumen of the catheter;
a distal manifold in fluid communication with at least one lumen of the catheter and in fluid communication with the bundle of hollow fiber membranes;
a proximal manifold in fluid communication with at least one lumen of the catheter and in fluid communication with the bundle of hollow fiber membranes;
proximal means for allowing rotation of the fiber bundle and having a plurality of bearings contained in a housing;
distal means for allowing rotation of the fiber bundle and having a plurality of bearings contained in a housing; and
an external fitting in fluid communication with at least one lumen of the catheter and configured with couplings for introduction and removal of blood gasses.

2. The percutaneous respiratory assist catheter of claim 1, further comprising a cage surrounding the fiber bundle.

3. The percutaneous respiratory assist catheter of claim 2, wherein the cage is formed from a wire loom.

4. The percutaneous respiratory assist catheter of claim 3, wherein the cage is formed from nitinol.

5. The percutaneous respiratory assist catheter of claim 3, wherein the cage is formed from stainless steel.

6. The percutaneous respiratory assist catheter of claim 2, wherein the cage is formed from a coil.

7. The percutaneous respiratory assist catheter of claim 1, wherein the fiber bundle is configured for increased porosity.

8. The percutaneous respiratory assist catheter of claim 7, wherein the fiber bundle is configured from a fiber mat having a plurality of spacers.

9. The percutaneous respiratory assist catheter of claim 8, wherein the spacers are formed from polyurethane soaked felt strips.

10. A percutaneous respiratory assist catheter, comprising:
a catheter having a plurality of lumens;
a bundle of hollow fiber membranes'connected to the catheter and in fluid communication with at least one lumen of the catheter, wherein the fiber bundle is configured for increased porosity;
a cage surrounding the fiber bundle; and
means for providing rotation of the fiber bundle.

11. The percutaneous respiratory assist catheter of claim 10, wherein the cage is formed from a wire loom.

12. The percutaneous respiratory assist catheter of claim 10, wherein the cage is formed from a coil.

13. The percutaneous respiratory assist catheter of claim 10, wherein the cage is formed from nitinol.

14. The percutaneous respiratory assist catheter of claim 10, wherein the cage is formed from stainless steel.

15. The percutaneous respiratory assist catheter of claim 10, wherein the means for providing rotation of the fiber bundle includes means for varying the speed of rotation of the fiber bundle.

16. The percutaneous respiratory assist catheter of claim 10, wherein the means for providing rotation of the fiber bundle includes means for oscillating the direction of rotation of the fiber bundle.

17. A method for providing gas exchange in a blood vessel of a patient, comprising:
providing a percutaneous respiratory assist catheter configured with a catheter having a plurality of lumens, a bundle of hollow fiber membranes connected to the catheter and in fluid communication with at least one lumen of the catheter, a cage surrounding the fiber bundle, and means for providing rotation of the fiber bundle;
inserting the percutaneous respiratory assist catheter into a vessel of a patient;
supplying a flow of gas to a first lumen of the catheter; and
removing a flow of gas from a second lumen of the catheter.

18. The method for providing gas exchange of claim 17, wherein providing a percutaneous respiratory assist catheter includes configuring the fiber bundle for increased porosity.

19. The method for providing gas exchange of claim 17, wherein providing rotation of the fiber bundle includes means for varying the speed of rotation of the fiber bundle.

20. The method for providing gas exchange of claim 17, wherein providing rotation of the fiber bundle includes means for oscillating the direction of rotation of the fiber bundle.
